(19) United States
(12) Patent Application Publication
(10) Pub. No.: US 2009/0186086 A1
(43) Pub. Date: Jul. 23, 2009

(54) Solid multilayer oral dosage forms

(75) Inventors: Sabarinath Shankar, Monmouth Junction, NJ (US); Indranil Nandi, Mahwah, NJ (US); Shailesh Shah, Livingston, NJ (US)

Correspondence Address: FOLEY AND LARDNER LLP;SUITE 500, 3000 K STREET NW, WASHINGTON, DC, 20007, US

(73) Assignee: Par Pharmaceutical, Inc.

(21) Appl. No.: 12/007,985

(22) Filed: Jan. 17, 2008

(51) Int. Cl. A61K 9/32; A61K 31/4045; A61P 29/00

(52) U.S. Cl. 424/482; 514/415

(57) ABSTRACT

Provided are multilayer compressed oral dosage forms comprising naproxen or a pharmaceutically acceptable salt thereof and sumatriptan or a pharmaceutically acceptable salt thereof. Processes of making and using the oral dosage forms also are described.

### FIELD OF INVENTION

The present invention relates generally to the field of pharmaceutical compositions for oral administration, including multilayer oral dosage forms containing sumatriptan and naproxen.

### BACKGROUND

An area of current research focus in the pharmaceutical industry is the development of oral dosage forms containing multiple active ingredients. Such dosage forms obviate certain problems associated with traditional methods for administering multiple drugs, such as non-compliance of patients with a prescribed medication schedule and the need for patients to take multiple medications. Different active ingredients may interact with one another, however, presenting challenges to preparing a single oral dosage form containing multiple active ingredients.

Naproxen is a non-steroidal anti-inflammatory drug (NSAID) with analgesic, antipyretic, and anti-inflammatory effects that reduce pain, fever, and inflammation. Naproxen and its pharmaceutically acceptable salts are used to treat pain, such as headache pain, including pain associated with a migraine headache. Naproxen sodium is available for over-the-counter sale in the United States, however it is available only by prescription in much of the world.

Sumatriptan is a triptan drug with a sulfonamide group that is commonly used to treat headache pain, especially pain associated with a migraine headache. Sumatriptan is a mid-line attack for mid-level to severe migraines, and is typically used on migraines that do not respond to NSAIDs.

U.S. Pat. Nos. 5,872,145 and 6,586,458 describe combination therapies in which triptan drugs are combined with NSAIDs, such as sumatriptan and naproxen, to improve pain relief in migraine patients, and a dosage form comprising a triptan and an NSAID. U.S. Pat. Nos. 6,926,907 and 7,030,162 and published U.S. Patent Applications 2006/0178349 and 2007/0207200 describe multilayer tablets including a triptan and an NSAID, wherein the triptan is released first, and the NSAID release is delayed by several minutes.

### SUMMARY

In accordance with one embodiment, the invention provides a compressed solid oral dosage form comprising a first layer and a second layer, wherein the first layer comprises naproxen or a pharmaceutically acceptable salt thereof and sumatriptan or a pharmaceutically acceptable salt thereof, and the second layer comprises naproxen or a pharmaceutically acceptable salt thereof. The ratio of the amount of naproxen or pharmaceutically acceptable salt thereof present in the first layer to the amount of naproxen or pharmaceutically acceptable salt thereof present in the second layer is from about 11:89 to 30:70, based on the total amount of naproxen or pharmaceutically acceptable salt thereof present in the oral dosage form, and the first layer comprises substantially all of the sumatriptan or pharmaceutically acceptable salt thereof present in the oral dosage form.

In specific embodiments, upon oral administration of the dosage form, the sumatriptan or pharmaceutically acceptable salt thereof dissolves independently of the naproxen or pharmaceutically acceptable salt thereof.

In one specific embodiment, the oral dosage form comprises naproxen sodium and sumatriptan succinate. The oral dosage form may comprise about 500 mg naproxen sodium. The oral dosage form may comprise about 119 mg sumatriptan succinate.

In some embodiments, the first layer comprises an intragranular portion and an extragranular portion, the intragranular portion comprising granulated naproxen or pharmaceutically acceptable salt thereof, and the extragranular portion comprising sumatriptan or a pharmaceutically acceptable salt thereof. The granulated naproxen or pharmaceutically acceptable salt thereof may be coated with polyvinyl pyrrolidone. In some embodiments, the intragranular portion of the first layer comprises one or more additives selected from the groups consisting of sodium carbonate, microcrystalline cellulose, polyvinyl pyrrolidone, and combinations thereof. In some embodiments, the extragranular portion of the first layer further comprises one or more additives selected from the groups consisting of lactose, microcrystalline cellulose, croscarmellose sodium, talc, magnesium stearate, and combinations thereof.

In some embodiments, the second layer comprises an intragranular portion and an extragranular portion, the intragranular portion comprising granulated naproxen or pharmaceutically acceptable salt thereof. The intragranular portion of the second layer may further comprise one or more additives selected from the groups consisting of microcrystalline cellulose, polyvinyl pyrrolidone, and combinations thereof. In some embodiments, the extragranular portion of the second layer comprises one or more additives selected from the groups consisting of microcrystalline cellulose, croscarmellose sodium, talc, magnesium stearate, and combinations thereof.

In some embodiments, the first layer comprises an intragranular portion and an extragranular portion, the second layer comprises an intragranular portion and an extragranular portion, and at least one of the intragranular portion of the first layer and the intragranular portion of second layer comprises croscarmellose sodium.

In some embodiments, the dosage form is a bilayer tablet with a hardness of about 12-26 kp.

In some embodiments, the first layer comprises a first planar surface and the second layer comprises a second planar surface, and the first planar surface of the first layer is adjacent the second planar surface of the second layer.

The invention also provides a process of preparing a compressed solid oral dosage form as described above, wherein the process comprises: (a) granulating naproxen or a pharmaceutically acceptable salt thereof to form a first intragranular portion, (b) blending the first intragranular portion with a first extragranular portion comprising sumatriptan or a pharmaceutically acceptable salt thereof, to form a first layer blend, (c) granulating naproxen or a pharmaceutically acceptable salt thereof to form a second intragranular portion, (d) blending the second intragranular portion with a second extragranular portion comprising one or more additives, to form a second layer blend, and (e) compressing the first layer blend and the second layer blend to form the oral dosage form.

In some embodiments, step (a) comprises wet granulation with sodium carbonate and a polymer binder. In some embodiments, step (a) results in the formation of granulated naproxen or pharmaceutically acceptable salt thereof coated with polymer binder. In some embodiments, the polymer binder comprises polyvinyl pyrrolidone.

The invention also provides an oral dosage form made by the process described above.

Both the foregoing general description and the following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates the dissolution profile of sumatriptan in a pH 7.4 solution.

FIG. 2 illustrates the dissolution profile of naproxen in a pH 7.4 solution.

FIG. 3 illustrates the dissolution profile of sumatriptan in a pH 4.5 solution.

FIG. 4 illustrates the dissolution profile of naproxen in a pH 4.5 solution.

FIG. 5 illustrates the dissolution profile of sumatriptan in a pH 2.0 solution.

FIG. 6 illustrates the dissolution profile of naproxen in a pH 2.0 solution.

FIG. 7 illustrates a process flow chart of an exemplary method of making a pharmaceutical composition according to the present invention.

### DETAILED DESCRIPTION

The present invention provides novel pharmaceutical compositions for oral administration of naproxen and sumatriptan. One aspect of the present invention relates to the fact that naproxen is poorly soluble in lower pH conditions (such as the upper digestive tract) and forms a gel-like matrix in the stomach. This gel-like naproxen matrix retards the dissolution of other co-administered drugs, such as sumatriptan, thereby reducing efficacy. The present invention solves this problem by providing a composition comprising naproxen and sumatriptan wherein the sumatriptan dissolves independently of the naproxen. Thus, in some embodiments the invention provides multilayer oral dosage forms comprising a first layer comprising naproxen or a pharmaceutically acceptable salt thereof and sumatriptan or a pharmaceutically acceptable salt thereof, and a second layer comprising naproxen or a pharmaceutically acceptable salt thereof. In accordance with some embodiments, the first layer comprises substantially all or all of the sumatriptan (or pharmaceutically acceptable salt thereof) present in the dosage form.

### A. Definitions

Unless defined otherwise, the terms used herein are intended to have their ordinary meaning in the art.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10% of the particular term.

Unless otherwise specified, “a,” “an” or “the” designates one or more, and words used in the singular also include the plural.

As used herein, “compressed” dosage form (e.g., “compressed tablet”), refers to a dosage form comprising a compressed powder. For example, a compressed tablet may be formed using a rotary tablet press or other similar machinery known to one of skill in the art.

As used herein, “bilayer” compressed dosage form (e.g., “bilayer tablet”) refers to a single compressed dosage form comprising two layers. A bilayer compressed dosage form can be made in a single compression step.

As used herein, “wet granulation” refers to a process known in the pharmaceutical arts that involves forming granules by the addition of a liquid, such as purified water, alcohol, or a binder solution.

For the sake of convenience, unless otherwise specified, “naproxen” as used herein refers to naproxen and its pharmaceutically acceptable salts, and “sumatriptan” as used herein refers to sumatriptan and its pharmaceutically acceptable salts.

### B. Oral Dosage Forms

As noted above, one aspect of the present invention provides a compressed solid oral dosage form comprising a first layer and a second layer, wherein the first layer comprises naproxen (or a pharmaceutically acceptable salt thereof) and sumatriptan (or a pharmaceutically acceptable salt thereof) and the second layer comprises naproxen (or a pharmaceutically acceptable salt thereof).

In some embodiments, the ratio of the amount of naproxen present in the first layer to the amount of naproxen present in the second layer is from about 11:89 to 30:70, including about 11:89, about 15:85, about 20:80, about 25:75, and about 30:70, based on the total amount of naproxen present in the oral dosage form, and the first layer comprises substantially all of the sumatriptan or present in the oral dosage form.

In some embodiments, the first layer comprises an intragranular portion and an extragranular portion, with the intragranular portion comprising naproxen, and the extragranular portion comprising sumatriptan. In some embodiments, the second layer comprises an intragranular portion and an extragranular portion, with the intragranular portion comprising naproxen, and the extragranular portion comprising pharmaceutically acceptable additives.

In some embodiments, the naproxen is naproxen sodium. In some embodiments, the dosage form comprises about 250 mg, about 375 mg, or about 500 mg naproxen sodium.

In some embodiments, the sumatriptan is sumatriptan succinate. In some embodiments, the dosage form comprises about 25 mg, about 50 mg, about 100 mg, about 119 mg or about 120 mg sumatriptan succinate.

In some embodiments, the dosage form comprises about 250 mg, about 375 mg, or about 500 mg naproxen sodium and about 25 mg, about 50 mg, about 100 mg, about 119 mg or about 120 mg sumatriptan succinate. In some embodiments, the dosage form comprises about 500 mg naproxen sodium, and about 119 mg sumatriptan succinate.

Another aspect of the invention provides a process of preparing such compressed solid oral dosage forms. In some embodiments, the process comprises forming a first layer blend and a second layer blend and compressing the first and second layer blends to form a bilayer compressed solid oral dosage form. The first layer blend may be prepared by (a) granulating naproxen to form a first intragranular portion, (b) blending the first intragranular portion with a first extragranular portion comprising sumatriptan to form a first layer blend. The second layer blend may be prepared by (c) granulating naproxen to form a second intragranular portion, (d) blending the second intragranular portion with a second extragranular portion comprising one or more additives to form a second layer blend. As stated above, the first layer blend and the second layer blend may be compressed to form the oral dosage form.

1. First Layer

The first layer can be made my any means known in the art.

(a) Intragranular Portion of the First Layer

In embodiments where the first layer comprises an intragranular portion comprising naproxen, the naproxen can be granulated using methods and equipment that are well-known in the art. In embodiments where the first layer comprises an extragranular portion comprising sumatriptan, the granulated naproxen can be blended with sumatriptan to form a first layer blend.

For example, the naproxen can be granulated by wet granulation in purified water or with a binder solution. The wet granulation can be carried out by any means known in the art, such as in a high shear granulator or a fluid bed granulator. The wet granules can be dried to form dried granules, such as by drying in a hot pack oven or a fluid bed dryer. The dried granules can be milled by any means known in the art, such as using hammer mills/mechanical impact mills. In some embodiments, the hammer mills/mechanical impact mills are Fitzpatrick mills. In some embodiments, the hammer mills/mechanical impact mills are used with screen #0050 or another screen having a pore size of about 0.050 inches/1.27 millimeters. Mixing or blending can be achieved using conventional blenders, such as v-blenders or double cone blenders.

The intragranular portion of the first layer may include pharmaceutically acceptable additives, such as one or more of the disintegrants, binders, fillers, diluents, lubricants, and glidants discussed in more detail below. Such additives may be processed with the naproxen, e.g., subject to the same granulation, drying and milling steps, or may be added to the intragranular portion at any stage in the process.

In some embodiments, the intragranular portion of the first layer comprises naproxen in an amount of about 12-20% w/w, including about 15-17% w/w, based on the total weight of the first layer.

In some embodiments, the intragranular portion of the first layer comprises a pharmaceutically acceptable salt, such as a pharmaceutically acceptable basic salt. Examples of suitable salts include calcium carbonate, potassium carbonate, sodium carbonate, sodium bicarbonate, and potassium bicarbonate. In some embodiments, the intragranular portion of the first layer comprises sodium carbonate. In some embodiments, the intragranular portion of the first layer comprises the basic salt, such as sodium carbonate, in an amount of about 1-8% w/w, including about 3-5% w/w, based on the total weight of the first layer.

In some embodiments, the intragranular portion of the first layer comprises a binder. In specific embodiments, the intragranular portion of the first layer comprises polyvinyl pyrrolidone as a binder. In some embodiments, the intragranular portion of the first layer comprises polyvinyl pyrrolidone in an amount of about 3-10% w/w, including about 4-6% w/w, based on the total weight of the first layer.

In embodiments where the intragranular portion is formed by a process comprising wet granulation of naproxen with a polymer binder (such as polyvinyl pyrrolidone), the resulting naproxen granules are coated with polymer binder, e.g., the naproxen granules are uniformly coated with binder.

(b) Extragranular Portion of the First Layer

As noted above, in some embodiments, the first layer may comprise an extragranular portion that comprises sumatriptan. The extragranular portion of the first layer may include pharmaceutically acceptable additives, such as one or more of the disintegrants, binders, fillers, diluents, lubricants, and glidants discussed in more detail below. Such additives may be blended with the sumatriptan, or may be added when the sumatriptan is blended with the intragranular portion.

In some embodiments, the extragranular portion of the first layer comprises sumatriptan in an amount of about 15-23% w/w, including 18-20% w/w, based on the total weight of the first layer.

In some embodiments, the first layer comprises an intragranular portion and an extragranular portion, wherein the intragranular portion comprises naproxen, diluents or fillers, and binders, and the extragranular portion comprises sumatriptan, diluents or fillers, disintegrants, glidants, and lubricants.

In accordance with specific embodiments, the first layer comprises an intragranular portion and an extragranular portion, wherein the intragranular portion comprises naproxen sodium, microcrystalline cellulose, polyvinyl pyrrolidone, and sodium carbonate, and the extragranular portion comprises: sumatriptan succinate, spray-dried lactose with microcrystalline cellulose, croscarmellose sodium, talc, and magnesium stearate.

For example, the first layer intragranular portion may comprise about 12-20% w/w naproxen sodium, about 20% to 60% w/w microcrystalline cellulose, about 3-10% w/w polyvinyl pyrrolidone, and about 1-8% w/w sodium carbonate, and the first layer extragranular portion may comprise about 15-23% w/w sumatriptan succinate, about 20% to 60% w/w spray-dried lactose with microcrystalline cellulose, about 2% to 10% w/w croscarmellose sodium, about 1% to 7% w/w talc, and about 0.5% to 2% w/w magnesium stearate, all based on the total weight of the first layer.

In accordance with specific embodiments, the first layer comprises an intragranular portion and an extragranular portion, wherein the intragranular portion comprises: about 15.4% w/w naproxen sodium, about 24.5% w/w microcrystalline cellulose, about 5% w/w polyvinyl pyrrolidone, and about 3.7% w/w sodium carbonate, and the extragranular portion comprises about 18.3% w/w sumatriptan succinate, about 26.1% w/w spray-dried lactose with microcrystalline cellulose, about 4% w/w croscarmellose sodium, about 2% w/w talc, and about 1% w/w magnesium stearate, all based on the total weight of the first layer.

In specific embodiments, the first layer intragranular portion is blended with the first layer extragranular portion, to form a first layer blend. The first layer blend may be compressed into a solid oral dosage form, as discussed in more detail below.

2. Second Layer

The second layer can be made by any means known in the art.

(a) Intragranular Portion of the Second Layer

In embodiments where the second layer comprises an intragranular portion comprising naproxen, the naproxen can be granulated using methods and equipment that are well-known in the art. In embodiments where the second layer comprises an extragranular portion comprising pharmaceutically acceptable additives, the granulated naproxen can be blended with the additives.

The intragranular portion of the second layer can be prepared as described above with reference to the first layer, such as, for example, by wet granulation, drying, and milling. The wet granulation may be effected in purified water or in a binder solution, as discussed above.

The intragranular portion of the second layer may include pharmaceutically acceptable additives, such as one or more of the disintegrants, binders, fillers, diluents, lubricants, and glidants discussed in more detail below. Such additives may be processed with the naproxen or may be added to the intragranular portion at any stage in the process.

In some embodiments, the intragranular portion of the second layer comprises naproxen in an amount of about 50-75% w/w, including about 55-65% w/w, based on the total weight of the second layer.

In some embodiments, the intragranular portion of the second layer comprises a pharmaceutically acceptable salt, such as a pharmaceutically acceptable basic salt, as discussed above. In some embodiments, the intragranular portion of the second layer comprises sodium carbonate. In some embodiments, the intragranular portion of the second layer comprises the basic salt, such as sodium carbonate, in an amount of about 1-8% w/w, including about 3-5% w/w, based on the total weight of the second layer.

In some embodiments, the intragranular portion of the second layer comprises a binder. In specific embodiments, the intragranular portion of the second layer comprises polyvinyl pyrrolidone as a binder. In some embodiments, the intragranular portion of the second layer comprises polyvinyl pyrrolidone in an amount of about 3-10% w/w, including about 4-6% w/w, based on the total weight of the second layer.

As discussed above, in embodiments where the intragranular portion is formed by a process comprising wet granulation of naproxen with a polymer binder (such as polyvinyl pyrrolidone), the resulting naproxen granules are coated with polymer binder, e.g., the naproxen granules are uniformly coated with binder.

(b) Extragranular Portion of the Second Layer

As noted above, in some embodiments, the second layer may comprise an extragranular portion that comprises pharmaceutically acceptable additives, such as one or more of the disintegrants, binders, fillers, diluents, lubricants, and glidants discussed in more detail below. Such additives may be blended together prior to blending with the intragranular portion, or may be directly blended with the intragranular portion.

In some embodiments, the second layer comprises an intragranular portion and an extragranular portion, wherein the intragranular portion comprises naproxen, diluents or fillers, and binders, and the extragranular portion comprises diluents or fillers, disintegrants, glidants, and lubricants.

In specific embodiments, the second layer comprises an intragranular portion and an extragranular portion, wherein the intragranular portion comprises naproxen sodium, microcrystalline cellulose, and polyvinyl pyrrolidone, and the extragranular portion comprises microcrystalline cellulose, croscarmellose sodium, talc, and magnesium stearate.

For example, the second layer intragranular portion may comprise about 50-75% w/w naproxen sodium, about 20% to 60% w/w microcrystalline cellulose, and about 3-10% w/w polyvinyl pyrrolidone, and the second layer extragranular portion may comprise about 20% to 60% w/w microcrystalline cellulose, about 2% to 10% w/w croscarmellose sodium, about 1% to 7% w/w talc, and about 0.5% to 2% w/w magnesium stearate, all based on the total weight of the first layer.

In specific embodiments, the second layer comprises an intragranular portion and an extragranular portion, wherein the intragranular portion comprises about 66.7% w/w naproxen sodium, about 8.8% w/w microcrystalline cellulose, and about 3.9% w/w polyvinyl pyrrolidone, and the extragranular portion comprises about 13% w/w microcrystalline cellulose, about 2.2% w/w croscarmellose sodium, about 4.5% w/w talc, and about 0.8% w/w magnesium stearate, all based on the total weight of the second layer.

In specific embodiments, the second layer intragranular portion is blended with the second layer extragranular portion, to form a second layer blend. The second layer blend may be compressed into a solid oral dosage form, as discussed in more detail below.

3. Optional Additives

In addition to naproxen and sumatriptan (or their pharmaceutically acceptable salts), the oral dosage forms described herein may comprise one or more additional pharmaceutically acceptable additives. For example, the dosage form may include one or more disintegrants, fillers, binders, diluents, anti-adherents, lubricants, glidants, or any other pharmaceutically acceptable additives. These additives may be chosen for their effect on the dissolution of the naproxen and sumatriptan, or may be selected for any number of other reasons known to those skilled in the art.

Various embodiments of the invention may include a pharmaceutically acceptable disintegrant. Disintegrants can be used to provide any of several advantageous characteristics to the compositions, and facilitate the breaking up of the compacted oral dosage form when added to a liquid environment, e.g., upon oral administration. In embodiments prepared using wet granulation, a disintegrant is advantageously included in the intragranular portion. Alternatively, a disintegrant may be added to both the intragranular portion and the extragranular portion. Specific examples of disintegrants include, but are not limited to, croscarmellose sodium, starch, starch derivatives, corn starch, potato starch, maize starch, modified starches, clays, gums, cellulose, cellulose derivates, alginates, crosslinked polyvinyl pyrrolidone, sodium starch glycolate, microcrystalline cellulose, cross-povidone, sodium starch glycolate, and mixtures thereof. In specific embodiments, a disintegrant is selected from the group consisting of crosslinked polyvinyl pyrrolidone (crospovidone), croscarmellose sodium, sodium starch glycolate, alginic acid, and sodium alginate.

In some embodiments, the disintegrant is croscarmellose sodium. In specific embodiments, croscarmellose sodium is present in a range of about 2% to 10% w/w, based on the total weight of the layer.

Various embodiments of the invention may include pharmaceutically acceptable binders (adhesives). Binders are agents that impart cohesive properties to powdered materials through particle-particle bonding. Examples of suitable binders include celluloses and crosslinked polyvinyl pyrrolidone, matrix binders (dry starch, dry sugars), film binders (polyvinyl pyrrolidone (PVP), starch paste, celluloses, bentonite, sucrose), and chemical binders (polymeric cellulose derivatives, such as carboxy methyl cellulose, hydroxypropylcellulose (HPC) and hydroxypropylmethylcellulose (HPMC); sugar syrups; corn syrup; water soluble polysaccharides such as acacia, tragacanth, guar and alginates; gelatin; gelatin hydrolysate; agar; sucrose; dextrose; and non-cellulosic binders, such as polyvinyl pyrrolidone, polyethylene glycol (PEG), vinyl pyrrolidone copolymers, pregelatinized starch, sorbitol, glucose, microcrystalline cellulose, such as FMC BioPolymer's Avicel® PH101 and Avicel® PH102, and silicified microcrystalline cellulose, such as Penwest Pharmaceutical's ProSolv SMCC™). In specific embodiments, a binder is selected from the group consisting of corn starch, potato starch, polyvinyl pyrrolidone, hydroxypropylmethyl cellulose, and hydroxylpropyl cellulose. A binder may be included in any portion of the dosage form, such as the intragranular portion and/or extragranular portion of either or both of the first and second layers.

In some embodiments, the oral dosage form further comprises a pharmaceutically acceptable diluent or filler. Pharmaceutically acceptable diluents include, but are not limited to, lactose (such as lactose monohydrate, lactose anhydrous, and DMV International's Pharmatose® DCL21 crystalline alpha monohydrate milled lactose), mannitol, talc, magnesium stearate, sodium chloride, potassium chloride, citric acid, spray-dried lactose, starch, hydrolyzed starches, directly compressible starch, microcrystalline cellulose (such as Avicel® PH101 and Avicel® PH102), cellulosics, sorbitol, sucrose, glucose, sucrose-based materials, saccharides, calcium sulfate, dibasic calcium phosphate (such as Emcompress®) and dextrose, and/or mixtures of any of the foregoing. In specific embodiments, a diluent is selected from the group consisting of microcrystalline cellulose, lactose, mannitol, dicalcium phosphate, dextrose, compressible sugar, and spray-dried lactose with microcrystalline cellulose. A diluent may be may be included in any portion of the dosage form, such as the intragranular portion and/or extragranular portion of either or both of the first and second layers.

In some embodiments, the oral dosage form comprises magnesium stearate. In specific embodiments, the magnesium stearate is present in a range of about 0.5% to 2% w/w, based on the total weight of the layer.

In some embodiments, the diluent is microcrystalline cellulose or microlac (spray-dried lactose with microcrystalline cellulose). In specific embodiments, the microcrystalline cellulose or microlac is present in a range of about 20% to 60% w/w, based on the total weight of the layer.

Various embodiments of the invention may include pharmaceutically acceptable anti-adherents (anti-sticking agents, glidants, flow promoters, lubricants) such as talc, colloidal silicon dioxide, such as Aerosil® 200, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, calcium stearate, silica gel, starch, hydrogenated vegetable oils, sodium benzoate, sodium acetate, leucine, PEG-4000, and magnesium lauryl sulfate. In specific embodiments, an anti-adherents is selected from glidants and lubricants. Suitable glidants include, but are not limited to, colloidal silicon dioxide (Aerosil®), magnesium trisilicate, talc, and tribasic calcium phosphate. Suitable lubricants include, but are not limited to magnesium, aluminum, calcium, zinc stearate, and talc. An anti-adherent may be included in any portion of the dosage form, such as the intragranular portion and/or extragranular portion of either or both of the first and second layers. In specific embodiments, an anti-adherent is included in the extragranular portion of the first layer and/or the extragranular portion of the second layer.

In some embodiments, the glidant is talc. In specific embodiments, talc is present in a range of about 1% to 7% w/w, based on the total weight of the of each layer.

4. Compressed Dosage Forms

One aspect of the present invention provides a compressed solid oral dosage form comprising a first layer and a second layer, i.e., a bilayer solid oral dosage form. Such a bilayer oral dosage form may be made by compressing a first layer composition, such as a first layer blend as described above, and a second layer composition, such as a second layer blend as described above. The first and second layer blends may be compressed into a bilayer solid oral dosage form, e.g., a bilayer tablet, in a single compression step. For example, compression can be carried out using bilayer tabletting machines, e.g. a Cadmach bilayer press (Cadmach Machinery Co. PVT. Ltd., India) or a tablet press with displacement monitoring such as the Courtoy-R292F (Courtroy, nv, Belgium).

Thus, a compressed solid oral dosage form can be made by a process comprising: (a) granulating naproxen to form a first intragranular portion, (b) blending the first intragranular portion with a first extragranular portion comprising sumatriptan to form a first layer blend, (c) granulating naproxen to form a second intragranular portion, (d) blending the second intragranular portion with a second extragranular portion comprising pharmaceutically acceptable additives to form a second layer blend, and (e) compressing the first layer blend and the second layer blend to form the oral dosage form.

The dosage forms can be made into any desired shape, such as an oval shape, using suitable punches, such as oval punches. The dosage form may also be of any other appropriate shape, such as round, cubic, cylindrical, spherical. The dosage form can be any suitable size. For example, the size of the compressed tablets may range from about 1 mm to about 6 mm. In some embodiments, the dose form comprises a first layer comprising a first planar surface and a second layer comprising a second planar surface, wherein the first planar surface of the first layer is adjacent the second planar surface of the second layer.

As would be appreciated by one skilled in the art, oral dosage forms of the present invention can be formulated to have any desired hardness. Hardness can be assessed by means commonly used in the art, for example, using commercially available hardness testers that are routinely used for assessing the hardness of pharmaceutical dosage forms. In one embodiment, the dosage form has a hardness of between about 12 kp to 26 kp.

### C. Therapeutic Methods

One aspect of the invention provides methods of treating pain, such as headache pain, including migraine, that comprises orally administering to a subject in need thereof a compressed solid oral dosage form as described herein. The subject may be any mammal, including humans, that is suffering from or at risk of developing pain, such as headache pain, including migraine.

In some embodiments, upon oral administration of the dosage form, the sumatriptan dissolves substantially immediately and independently of the naproxen. For example, following oral administration at pH 7.4, the sumatriptan may dissolve within 20 minutes or less, 10 minutes or less, or 5 minutes or less, and the naproxen may dissolve within 30 minutes or less, 20 minutes or less, or 10 minutes or less. In another example, following oral administration at pH 4.5, the sumatriptan may dissolve within 30 minutes or less, 20 minutes or less, 10 minutes or less, or 5 minutes or less, and the naproxen may dissolve within 20 minutes or less, 10 minutes or less, or 5 minutes or less. In another example, following administration at pH 2.0, the sumatriptan may dissolve within 10 minutes or less or 5 minutes or less, and the naproxen may dissolve within 10 minutes or less or 5 minutes or less.

### EXAMPLE 1

Compressed bilayer tablets comprising a first layer and a second layer were produced with the following distributions of naproxen and sumatriptan.

| | % | % | % | % |
|---|---|---|---|---|
| | Naproxen | Naproxen | Sumatriptan | Sumatriptan |
| | in First | in Second | in First | in Second |
| Tablet | Layer | Layer | Layer | Layer |
| RB-170-S035 | 20 | 80 | 100 | 0 |
| RB-170-S038 | 30 | 70 | 100 | 0 |
| RB-170-S021 | 80 | 20 | 20 | 80 |

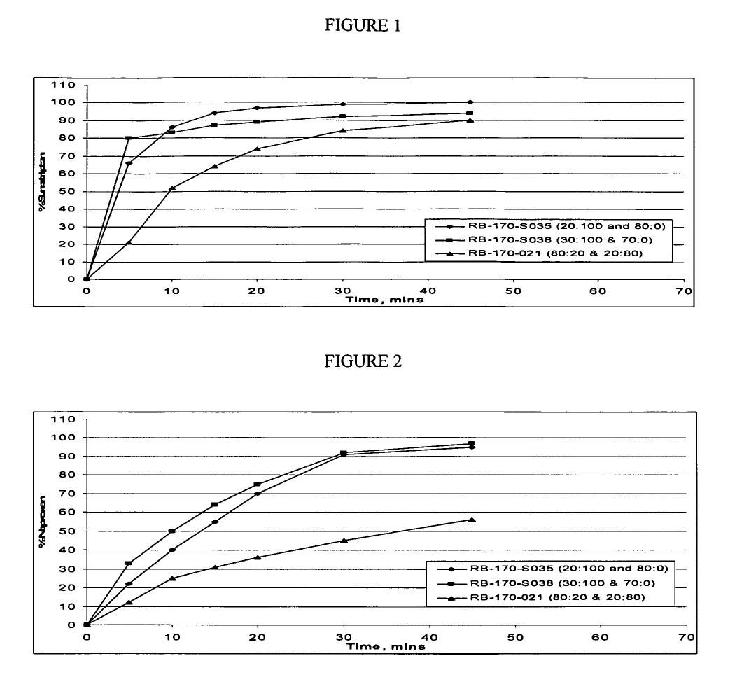

FIGS. 1 and 2 illustrate the dissolution profile of sumatriptan (FIG. 1) and naproxen (FIG. 2) from the tablets in 900 mL of a pH 7.4 phosphate buffer dissolution media in a #10 mesh basket at 50 rpm. With the RB-170-021 tablet, a naproxen gel matrix formed that entrapped the sumatriptan, decreasing its dissolution and release. In contrast, with the RB-170-S035 and RB-170-S038 tablets, sumatriptan dissolved and was released immediately and independent of naproxen.

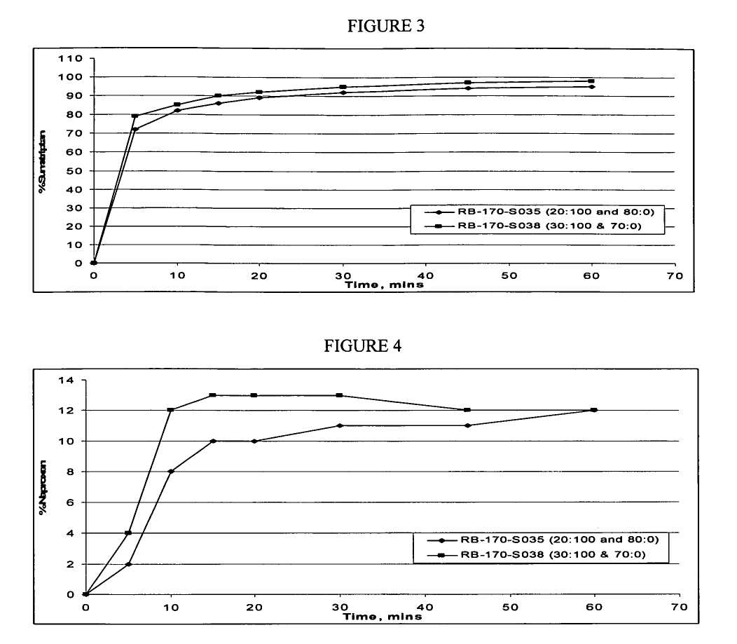

FIGS. 3 and 4 illustrate the dissolution profile of sumatriptan (FIG. 3) and naproxen (FIG. 4) from the RB-170-S035 and RB-170-S038 tablets in 900 mL of a pH 4.5 dissolution media in a #10 mesh basket at 50 rpm. As seen in FIG. 4, the solubility of naproxen is very low in lower pH conditions, as it forms a gel-like matrix. Nevertheless, the release profile of sumatriptan from the tablets is fast and complete, showing that sumatriptan is not entrapped in the naproxen gel matrix, but is released from these tablets independent of naproxen.

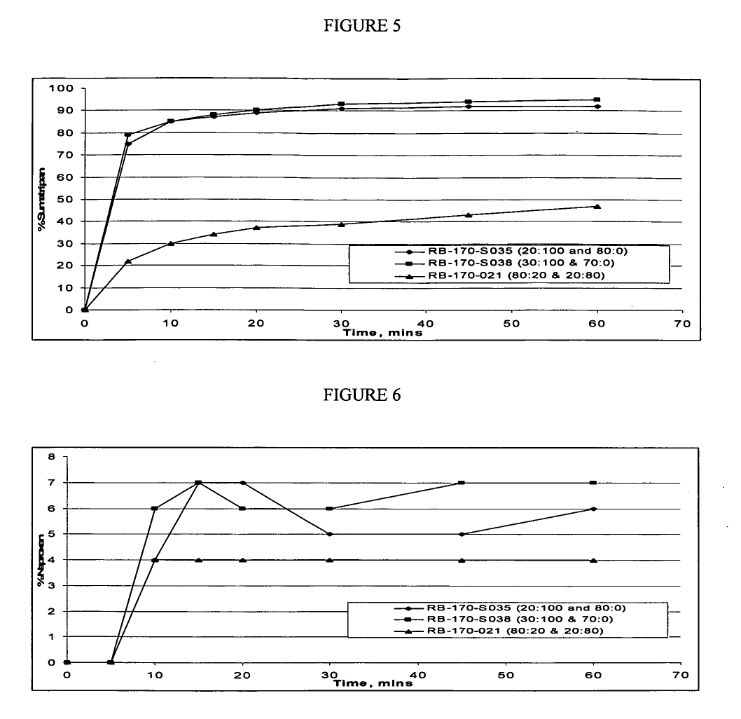

FIGS. 5 and 6 illustrate the dissolution profile of sumatriptan (FIG. 5) and naproxen (FIG. 6) from the tablets in 900 mL of a pH 2.0 dissolution media in a #10 mesh basket at 50 rpm. Naproxen is insoluble at this pH condition, as it forms gel-like matrix. In FIG. 5, the results for the RB-170-021 tablet reflect the entrapment of sumatriptan in the naproxen gel matrix, which decreases the sumatriptan release. In contrast, the results for the RB-170-S035 and RB-170-S038 tablets reflect that sumatriptan is released from those tablets immediately and independent of naproxen.

### EXAMPLE 2

A compressed bilayer tablet containing naproxen sodium and sumatriptan succinate is prepared by preparing a first layer blend and a second layer blend, and compressing the first and second layer blends into a bilayer tablet.

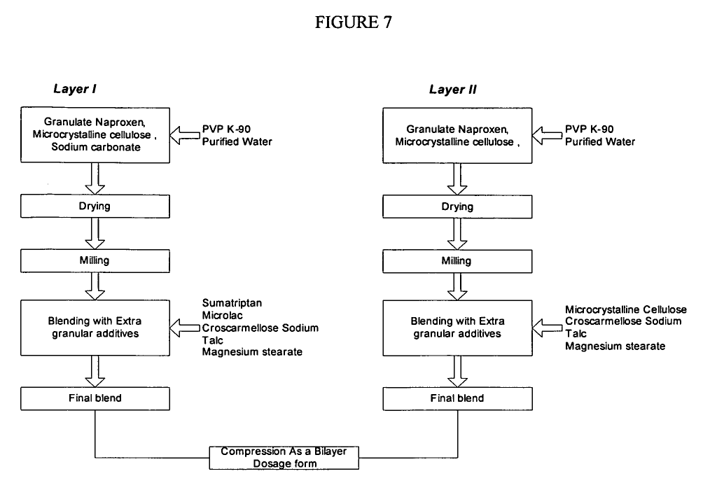

To prepare the first layer blend, an intragranular portion and an extragranular portion are prepared as described below and as depicted schematically in FIG. 7.

For the intragranular portion, naproxen sodium is placed in a high shear horizontal granulator with microcrystalline cellulose and sodium carbonate, and granulated using a binder solution comprising polyvinyl pyrrolidone and purified water, resulting in a uniform coating of polyvinyl pyrrolidone on the naproxen sodium granules. The resulting granules are dried in a fluid bed dryer and milled.

The intragranular portion is blended with an extragranular components including sumatriptan, Microlac (spray-dried lactose with microcrystalline cellulose), croscarmellose sodium, and talc. Magnesium stearate is added to form the first layer blend.

To prepare the second layer blend, an intragranular portion and an extragranular portion are prepared as follows:

For the intragranular portion, naproxen sodium is placed in a high shear horizontal granulator with microcrystalline cellulose, and granulated using a binder solution comprising polyvinyl pyrrolidone and purified water, resulting in a uniform coating of polyvinyl pyrrolidone on the naproxen sodium granules. The resulting granules are dried in a fluid bed dryer and milled.

The intragranular portion is blended with extragranular components including microcrystalline cellulose, croscarmellose sodium, and talc. Magnesium stearate is added to form the second layer blend.

To prepare the bilayer tablet, the first layer blend and the second layer blend are compressed in a single compression step.

Table 1 provides the formulation of the compressed bilayer tablet.

TABLE 1

| Formula First Layer | Qty/Unit, mg | % | Formula Second Layer | Qty/Unit, mg | % |
|---|---|---|---|---|---|
| Intragranular portion: | | | Intragranular portion: | | |
| Naproxen Sodium | 100.00 | 15.4 | Naproxen Sodium | 400.00 | 66.7 |
| Microcrystalline Cellulose | 159.10 | 24.5 | Microcrystalline | 52.95 | 8.8 |
| | | | Cellulose | | |
| Polyvinyl Pyrrolidone | 32.50 | 5.0 | Polyvinyl Pyrrolidone | 23.60 | 3.9 |
| Sodium Carbonate | 24.00 | 3.7 | Purified Water | Q.S | — |
| Purified Water | Q.S | — | | | |
| Extragranular portion: | | | Extragranular portion: | | |
| Sumatriptan Succinate | 119.00 | 18.3 | Microcrystalline | 77.95 | 13.0 |
| | | | Cellulose | | |
| Microlac | 169.90 | 26.1 | Croscarmellose Sodium | 13.50 | 2.2 |
| Croscarmellose Sodium | 26.00 | 4.0 | Talc | 27.00 | 4.5 |
| Talc | 13.00 | 2.0 | Magnesium Stearate | 5.00 | 0.8 |
| Magnesium Stearate | 6.50 | 1.0 | | | |
| Total: | 650.00 | | | 600.00 | |

The total tablet weight is 1250 mg, with a hardness of 15 kp.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A compressed solid oral dosage form comprising a first layer and a second layer, wherein:
the first layer comprises naproxen or a pharmaceutically acceptable salt thereof and sumatriptan or a pharmaceutically acceptable salt thereof;
the second layer comprises naproxen or a pharmaceutically acceptable salt thereof;
the ratio of the amount of naproxen or pharmaceutically acceptable salt thereof present in the first layer to the amount of naproxen or pharmaceutically acceptable salt thereof present in the second layer is from about 11:89 to 30:70, based on the total amount of naproxen or pharmaceutically acceptable salt thereof present in the oral dosage form, and
the first layer comprises substantially all of the sumatriptan or pharmaceutically acceptable salt thereof present in the oral dosage form.

2. The oral dosage form of claim 1, wherein, upon oral administration of the dosage form, the sumatriptan or pharmaceutically acceptable salt thereof dissolves independently of the naproxen or pharmaceutically acceptable salt thereof.

3. The oral dosage form of claim 1, wherein the first layer further comprises sodium carbonate.

4. The oral dosage form of claim 1, comprising naproxen sodium and sumatriptan succinate.

5. The oral dosage form of claim 1, comprising about 500 mg naproxen sodium.

6. The oral dosage form of claim 1, comprising about 119 mg sumatriptan succinate.

7. The oral dosage form of claim 5, comprising about 119 mg sumatriptan succinate.

8. The oral dosage form of claim 1, wherein the first layer comprises all of the sumatriptan or pharmaceutically acceptable salt thereof that is present in the dosage form.

9. The oral dosage form of claim 1, wherein the first layer comprises an intragranular portion and an extragranular portion, the intragranular portion comprising granulated naproxen or pharmaceutically acceptable salt thereof, and the extragranular portion comprising sumatriptan or a pharmaceutically acceptable salt thereof.

10. The oral dosage form of claim 9, wherein the intragranular portion of the first layer further comprises one or more additives selected from the group consisting of sodium carbonate, microcrystalline cellulose, polyvinyl pyrrolidone, and combinations thereof.

11. The oral dosage form of claim 9, wherein the intragranular portion of the first layer further comprises sodium carbonate in an amount of about 1-8% w/w, based on the total weight of the first layer.

12. The oral dosage form of claim 9, wherein the intragranular portion of the first layer further comprises polyvinyl pyrrolidone in an amount of about 3-10% w/w, based on the total weight of the first layer.

13. The oral dosage form of claim 9, wherein the granulated naproxen or pharmaceutically acceptable salt thereof is coated with polyvinyl pyrrolidone.

14. The oral dosage form of claim 9, wherein the extragranular portion of the first layer further comprises one or more additives selected from the groups consisting of lactose, microcrystalline cellulose, croscarmellose sodium, talc, magnesium stearate, and combinations thereof.

15. The oral dosage form of claim 1, wherein the second layer comprises an intragranular portion and an extragranular portion, the intragranular portion comprising granulated naproxen or pharmaceutically acceptable salt thereof.

16. The oral dosage form of claim 15, wherein the intragranular portion of the second layer further comprises one or more additives selected from the groups consisting of microcrystalline cellulose, polyvinyl pyrrolidone, and combinations thereof.

17. The oral dosage form of claim 15, wherein the extragranular portion of the second layer further comprises one or more additives selected from the groups consisting of microcrystalline cellulose, croscarmellose sodium, talc, magnesium stearate, and combinations thereof.

18. The oral dosage form of claim 1, wherein the first layer comprises an intragranular portion and an extragranular portion, the second layer comprises an intragranular portion and an extragranular portion, and at least one of the intragranular portion of the first layer and the intragranular portion of second layer comprises croscarmellose sodium.

19. The oral dosage form of claim 18, wherein the intragranular portion of the first layer comprises croscarmellose sodium in an amount of about 2-10% w/w, based on the total weight of the first layer, and the intragranular portion of the second layer comprises croscarmellose sodium in an amount of about 2-10% w/w, based on the total weight of the second layer.

20. The oral dosage form of claim 1, further comprising a pharmaceutically acceptable additive selected from the group consisting of disintegrants, binders, diluents, lubricants, glidants, and combinations thereof.

21. The oral dosage form of claim 20, comprising:
croscarmellose sodium in each of the first and second layers in an amount of from about 2-10% w/w, based on the weight of each layer;
microcrystalline cellulose or spray-dried lactose with microcrystalline cellulose in each of the first and second layers in an amount of from about 20-60% w/w, based on the weight of each layer;
magnesium stearate in each of the first and second layers in an amount of from about 0.5-2% w/w, based on the weight of each layer; and
talc in each of the first and second layers in an amount of from about 1-7% w/w, based on the weight of each layer.

22. The oral dosage form of claim 1, wherein the first layer comprises a first planar surface and the second layer comprises a second planar surface, and the first planar surface of the first layer is adjacent the second planar surface of the second layer.

23. The oral dosage form of claim 1, wherein the dosage form is a bilayer tablet with a hardness of about 12-26 kp.

24. A compressed solid oral dosage form comprising a first layer and a second layer, wherein:
the first layer comprises an intragranular portion and an extragranular portion, wherein the intragranular portion of the first layer comprises:
about 15.4% w/w naproxen sodium,
about 24.5% w/w microcrystalline cellulose,
about 5% w/w polyvinyl pyrrolidone, and
about 3.7% w/w sodium carbonate,
all based on the total weight of the first layer;
and the extragranular portion of the first layer comprises:
about 18.3% w/w sumatriptan succinate,
about 26.1% w/w spray-dried lactose with microcrystalline cellulose,
about 4% w/w croscarmellose sodium,
about 2% w/w talc, and
about 1% w/w magnesium stearate,
all based on the total weight of the first layer;
the second layer comprises an intragranular portion and an extragranular portion, wherein the intragranular portion of the second layer comprises:
about 66.7% w/w naproxen sodium,
about 8.8% w/w microcrystalline cellulose, and
about 3.9% w/w polyvinyl pyrrolidone,
all based on the total weight of the second layer;
and the extragranular portion of the second layer comprises:
about 13% w/w microcrystalline cellulose,
about 2.2% w/w croscarmellose sodium,
about 4.5% w/w talc, and
about 0.8% w/w magnesium stearate,
all based on the total weight of the second layer.

25. A process of preparing a compressed solid oral dosage form comprising a first layer which comprises naproxen or a pharmaceutically acceptable salt thereof and sumatriptan or a pharmaceutically acceptable salt thereof and a second layer which comprises naproxen or a pharmaceutically acceptable salt thereof, wherein the process comprises:
(a) granulating naproxen or a pharmaceutically acceptable salt thereof to form a first intragranular portion,
(b) blending the first intragranular portion with a first extragranular portion comprising sumatriptan or a pharmaceutically acceptable salt thereof, to form a first layer blend,
(c) granulating naproxen or a pharmaceutically acceptable salt thereof to form a second intragranular portion,
(d) blending the second intragranular portion with a second extragranular portion comprising providing one or more additives, to form a second layer blend,
(e) compressing the first layer blend and the second layer blend to form the oral dosage form.

26. The process of claim 25, wherein the ratio of the amount of naproxen or pharmaceutically acceptable salt thereof present in the first layer blend to the amount of naproxen or pharmaceutically acceptable salt thereof present in the second layer blend is from about 11:89 to 30:70, based on the total amount of naproxen or pharmaceutically acceptable salt thereof present in the oral dosage form, and the first layer blend comprises substantially all of the sumatriptan or pharmaceutically acceptable salt present in the oral dosage form.

27. The process of claim 25, wherein step (a) comprises wet granulation with sodium carbonate and a polymer binder.

28. The process of claim 27, wherein step (a) results in the formation of granulated naproxen or pharmaceutically acceptable salt thereof coated with polymer binder.

29. The process of claim 28, wherein the polymer binder comprises polyvinyl pyrrolidone.

30. An oral dosage form made by the process of claim 25.
